(19) United States
(12) Patent Application Publication
(10) Pub. No.: US 2021/0392952 A1
(43) Pub. Date: Dec. 23, 2021

(54) Dispenser And Complementary Vaporizer

(71) Applicant: JT International S.A., Geneva (CH)

(72) Inventors: Moritz Werny, München (DE); Layth Sliman Bouchuiguir, Bellevue (CH)

(73) Assignee: JT International S.A., Geneva (CH)

(21) Appl. No.: 17/282,913

(22) PCT Filed: Oct. 21, 2019
(86) PCT No.: PCT/EP2019/078612

§ 371 Date: Apr. 5, 2021

(30) Foreign Application Priority Data
Oct. 25, 2018 (EP) 18202523.9

(51) Int. Cl. A24F 40/20; A24F 40/46; A24F 40/53; B65D 83/04

(52) U.S. Cl. CPC A24F 40/20; B65D 83/0418; A24F 40/53; A24F 40/46

(57) ABSTRACT

A dispenser for dispensing solid or semi-solid vaporizable consumables to a vaporizer includes a first store for storing at least one solid or semi-solid vaporizable consumable therein, and a first lid. The first store includes an opening for dispensing a solid or semi-solid vaporizable consumable therethrough, and the first lid is arranged such that it closes the opening in the first store when in a closed position. Further, the dispenser is arranged such that, in use, engaging a vaporizer with the dispenser loads a heating chamber in the vaporizer with a solid or semi-solid vaporizable consumable stored within the first store by aligning the opening in the first store with an opening in the heating chamber.

### FIELD OF INVENTION

The present invention relates to a dispenser for dispensing solid or semi-solid vaporizable consumables to a complimentary vaporizer.

### BACKGROUND OF THE INVENTION

Aerosol generating devices such as electronic cigarettes are relatively well known and are becoming increasingly popular alternatives to traditional smoking devices and methods. Devices which heat, rather than burn, a substance to produce a vapor for inhalation have become a popular subset of aerosol generating devices with consumers in recent years.

One such device employs a heating system which utilizes electrical energy to heat a vapor generation substance held within a chamber of the device when a user activates the device, typically with the press of a button. Vapor is created as the substance is heated, and the device is configured such that the user may inhale the vapor.

Once the vaporizable substance has been consumed by the user, i.e. it is no longer possible to generate vapor from the substance, the substance must be ejected from the device in order for a new vaporizable substance to be incorporated into the device for consumption.

Typically, the aerosol device and the vaporizable substance are carried on a user's person, and the loading and unloading of the device is performed by hand. Touching the vaporizable substance is unpleasant for the user, which has meant that the vaporizable substances used in heat-not-burn devices are typically sheathed to minimize the user's contact with the vaporizable substance. This is not an elegant solution, and it typically means that a portion of the consumable introduced into the aerosol device is not actually vaporizable (i.e. the sheath).

As market adoption of aerosol generating devices continues a rapid growth, users are seeking more efficient manners to transport the vaporizable substances to be used with their aerosol device, and improved mechanisms for loading and unloading their device.

The present invention seeks to mitigate at least some of the above-mentioned problems.

### SUMMARY OF THE INVENTION

According to a first aspect of the invention, there is provided a dispenser for dispensing solid or semi-solid vaporizable consumables to a vaporizer, wherein the dispenser comprises: a first store for storing at least one solid or semi-solid vaporizable consumable therein, wherein the first store comprises an opening for dispensing a solid or semi-solid vaporizable consumable therethrough; and a first lid, wherein the first lid is arranged such that it closes the opening in the first store when in a closed position. Further, said dispenser according to the first aspect of the invention is arranged such that, in use, engaging a vaporizer with the dispenser loads a heating chamber in the vaporizer with a solid or semi-solid vaporizable consumable stored within the first store by aligning the opening in the first store with an opening in the heating chamber. By utilizing such an arrangement, the user does not need to load the vaporizer with a solid or semi-solid vaporizable consumable by hand, and it is not necessary to sheath the consumables.

In a first embodiment, the dispenser according to the first aspect of the invention further comprises a second store having a capacity for storing at least one solid or semi-solid vaporizable consumable, preferably wherein the second store has a capacity for storing at least the same number of solid or semi-solid vaporizable consumables as the first store. In this embodiment, the second store comprises an opening for receiving a solid or semi-solid vaporizable consumable therethrough, and the dispenser is arranged such that, in use, engaging a vaporizer with the dispenser to unload a solid or semi-solid vaporizable consumable stored within a heating chamber in the vaporizer aligns the opening in the second store with an opening in the heating chamber. By utilizing such an arrangement, the user can store unused solid or semi-solid vaporizable consumables and dispose of used consumables in one device without needing to directly touch said consumables.

Preferably, the first store and the second store of the first embodiment are conjoined such that the opening of the second store is at an end of the body of the conjoined stores distal to the opening of the first store. Further preferably, the conjoined stores of the first embodiment comprise a movable divider for defining the first store and the second store such that when the first store is full of unused solid or semi-solid vaporizable consumables the first store is larger than the second store, and when there are no unused solid or semi-solid vaporizable consumables within the first store the second store is larger than the first store. Even further preferably, the conjoined stores of the first embodiment comprise a movable divider for defining the first store and the second store such that when the first store is full of unused solid or semi-solid vaporizable consumables the movable divider closes the opening of the second store, and when there are no unused solid or semi-solid vaporizable consumables within the first store the movable divider closes the opening of the first store. Such an arrangement reduces the bulkiness of the dispenser for the user whilst also providing the benefit of two stores.

In an alternate preferred assembly, the first store and the second store of the first embodiment are separated by the first lid. Such an assembly reduces the number of lids required and simplifies the structure of the dispenser.

In a second embodiment, the dispenser according to the first aspect of the invention further comprises an opening for ejecting a solid or semi-solid vaporizable consumable therethrough. In this embodiment, the dispenser is arranged such that, in use, engaging a vaporizer with the dispenser to unload a solid or semi-solid vaporizable consumable stored within a heating chamber in the vaporizer aligns the opening in the dispenser with an opening in the heating chamber. Such an arrangement allows a used solid or semi-solid vaporizable consumable to be disposed of and not stored without needing to directly touch said consumable.

Preferably, the dispenser according to the first aspect of the invention and the additionally mentioned embodiments comprises a housing which defines a first cavity, said first cavity comprising the first store. Further preferably, the housing defines a second cavity, said second cavity comprising the second store. The housing may be made of any suitable material, for example ABS (Acrylonitrile butadiene styrene) and/or PEEK (Polyether ether ketone).

Preferably, the first store of the dispenser according to the first aspect of the invention and the additionally mentioned embodiments is suitable for storing unused solid or semi-solid vaporizable consumables. Further preferably, the body of the first store is made of ABS (Acrylonitrile butadiene styrene) and/or PEEK (Polyether ether ketone).

Preferably, the second store of the dispenser according to the first aspect of the invention's additionally mentioned embodiments is suitable for storing used solid or semi-solid vaporizable consumables. Further preferably, the body of the second store is made of ABS (Acrylonitrile butadiene styrene) and/or PEEK (Polyether ether ketone).

Preferably, the dispenser according to the first aspect of the invention's additionally mentioned embodiments further comprises a second lid, wherein the second lid is arranged such that it closes the opening in the second store or the opening in the dispenser when in a closed position.

Preferably, the first store and/or the second store of the dispenser according to the first aspect of the invention and the additionally mentioned embodiments are either independently or simultaneously detachable from the dispenser housing. This allows an empty first store to be replaced with a replenished first store and/or a full second store to be disposed of and replaced with an empty store with ease.

Preferably, the dispenser according to the first aspect of the invention and the additionally mentioned embodiments further comprises an indicator or a window for determining the number of solid or semi-solid vaporizable consumables stored in the first store.

Preferably, the first lid in the dispenser according to the first aspect of the invention and the additionally mentioned embodiments is arranged such that engaging a vaporizer with the dispenser and moving the vaporizer from a first relative position to a second relative position in relation to the dispenser biases the first lid to an open position. Further preferably, the first lid is biased to an open position by engaging the external body of the vaporizer with the first lid and applying force. When the first lid is in an open position, the opening in the first store is open.

Preferably, the first lid in the dispenser according to the first aspect of the invention and the additionally mentioned embodiments is arranged such that engaging a vaporizer with the dispenser and moving the vaporizer from the second relative position to the first relative position in relation to the dispenser biases the first lid to the closed position.

Preferably, the second lid in the dispenser according to the additionally mentioned embodiments is arranged such that engaging a vaporizer with the dispenser and moving the vaporizer from a third relative position to a fourth relative position in relation to the dispenser biases the second lid to an open position. Further preferably, the second lid is biased to an open position by engaging the external body of the vaporizer with the second lid and applying force. When the second lid is in an open position, the opening in the second store is open.

Preferably, the second lid in the dispenser according to the additionally mentioned embodiments is arranged such that engaging a vaporizer with the dispenser and moving the vaporizer from the fourth relative position to the third relative position in relation to the dispenser biases the first lid to the closed position.

Preferably, the dispenser comprises one or more sensors configured to detect when the dispenser loads the heating chamber in the vaporizer with the solid or semi-solid vaporizable consumable.

Preferably, the dispenser comprises a detectable element configured to be detected by a sensor of the vaporizer when the vaporizer is engaged with the dispenser.

According to a second aspect of the invention, there is provided a vaporizer for use in combination with a dispenser of the first aspect of the invention or the additionally mentioned embodiments, wherein the vaporizer comprises: a heating chamber for receiving and heating a solid or semi-solid vaporizable consumable to generate vapor therefrom, the heating chamber comprising an opening for receiving and ejecting a solid or semi-solid vaporizable consumable therethrough; and a third lid, wherein the third lid is arranged such that it closes the opening in the heating chamber when in a closed position. Further, said vaporizer according to the second aspect of the invention is arranged such that, in use, engaging the vaporizer with the dispenser loads the heating chamber with a solid or semi-solid vaporizable consumable stored within the first store by aligning the opening in the first store with the opening in the heating chamber to receive the consumable.

Preferably, the third lid of the vaporizer according to the second aspect of the invention is locked in its closed position until a corresponding switch on the vaporizer is activated, thereby allowing the third lid to open. Preferably, the switch is a push button or connected to thermal sensor which turns the switch on when the temperature of the heating chamber get enough cool down. This ensures that the third lid does not open until the heating chamber is at a safe temperature.

Preferably, the third lid in the vaporizer according to the second aspect of the invention and the additionally mentioned embodiments is arranged such that engaging the vaporizer with the dispenser and moving the vaporizer from a fifth relative position to a sixth relative position in relation to the dispenser biases the third lid to an open position. Further preferably, the third lid is biased to an open position by engaging the external body of the dispenser with the third lid and applying force. When the third lid is in an open position, the opening in the heating chamber is open.

Preferably, the third lid in the vaporizer according to the second aspect of the invention and the additionally mentioned embodiments is arranged such that engaging a vaporizer with the dispenser and moving the vaporizer from the sixth relative position to the fifth relative position in relation to the dispenser biases the third lid to the closed position.

Preferably, the vaporizer comprises one or more sensors configured to detect when the dispenser loads the heating chamber in the vaporizer with the solid or semi-solid vaporizable consumable.

Preferably, the one or more sensors of the vaporizer comprise a first sensor configured to detect a detectable element of the dispenser when the vaporizer is engaged with the dispenser.

Preferably, the one or more sensors of the vaporizer comprise a second sensor configured to detect that the third lid is in the closed position.

Preferably, the vaporizer comprises a control circuit configured to detect that the dispenser has loaded the heating chamber in response to the second sensor detecting that the third lid is in the closed position after the first sensor detects that the vaporizer is engaged with the dispenser.

Preferably, in the first aspect of the invention, when the vaporizer is in the second relative position with respect to the dispenser, the opening of the first store and the opening of the heating chamber are aligned.

Preferably, in the additionally mentioned embodiments of the first aspect of the invention, when the vaporizer is in the fourth relative position with respect to the dispenser, the opening of the second store and the opening of the heating chamber are aligned.

Preferably, in the second aspect of the invention, when the vaporizer is in the sixth relative position with respect to the dispenser, the opening of the first store and the opening of the heating chamber are aligned.

Preferably, wherein the fifth relative position of the vaporizer with respect to the dispenser in the second aspect of the invention corresponds with the first and third relative positions of the vaporizer with respect to the dispenser in the first aspect of the invention, and the sixth relative position of the vaporizer with respect to the dispenser in the second aspect of the invention corresponds with the second or fourth relative position of the vaporizer with respect to the dispenser in the first aspect of the invention.

The above-mentioned movements of the dispenser and the vaporizer between two relative positions outline the “open and close” functions of the dispenser and the vaporizer based on interactive movement; such an assembly simplifies the “open and close” action for the user such that they are not required to remember to actively close the doors of the dispenser and the vaporizer.

Preferably, in the dispenser according to the first aspect of the invention or the additionally mentioned embodiments and in the vaporizer according the second aspect of the invention or the additionally mentioned embodiments, each lid is biased in a closed position when no external force is being exerted upon said lids. Further preferably, wherein the means for biasing each lid in a closed position when no external force is being exerted upon said lids is a mechanical spring such as a compression spring, an extension spring, a torsion spring, a conical spring, a barrel spring, or a magazine spring. This arrangement essentially automates the loading and unloading of the heating chamber for the user, providing increased ease of use.

According to a third aspect of the invention, there is provided a solid or semi-solid vaporizable consumable supply system comprising a vaporizer and a dispenser according to the first and second aspects of the invention or the additionally mentioned embodiments, wherein the vaporizer and the dispenser are arranged such that, in use, engaging the vaporizer with the dispenser biases the first lid and the third lid into their respective open positions and aligns the opening in the heating chamber with the opening in the first store thereby allowing a solid or semi-solid vaporizable consumable stored within the first store to be received from the first store into the heating chamber. Preferably, wherein biasing the first lid and the third lid into their respective open positions occurs simultaneously.

In a first embodiment, the vaporizer and the dispenser according to the third aspect of the invention are arranged such that, in use, engaging the vaporizer with the dispenser to unload the heating chamber biases the second lid and the third lid into their respective open positions and aligns the opening in the heating chamber with the opening in the second store thereby allowing a solid or semi-solid vaporizable consumable stored within the heating chamber to be received from the heating chamber into the second store. Preferably, wherein biasing the second lid and the third lid into their respective open positions occurs simultaneously.

In a second embodiment, the vaporizer and the dispenser according the third aspect of the invention are arranged such that, in use, engaging the vaporizer with the dispenser to unload the heating chamber biases the first and/or second lid(s) and the third lid into their respective open positions and aligns the opening in the heating chamber with the opening in the dispenser thereby allowing a solid or semi-solid vaporizable consumable stored within the heating chamber to be ejected from the system. Preferably, wherein biasing the first and/or second lid(s) and the third lid into their respective open positions occurs simultaneously.

Preferably, the vaporizer and the dispenser according to the third aspect of the invention and the additionally mentioned embodiments are arranged such that a solid or semi-solid vaporizable consumable may be received from the first store into the heating chamber by gravity.

Preferably, the vaporizer and the dispenser according to the third aspect of the invention and the additionally mentioned embodiments are arranged such that a solid or semi-solid vaporizable consumable may be received from the heating chamber into the second store or ejected from the system by gravity.

The term “solid or semi-solid vaporizable consumable” has its usual meaning in the art (i.e. a solid or highly viscous substance which can be vaporized and consumed). Example types of vapor generating solids include powder, granules, pellets, shreds, strands, porous material, foam material or sheets. Example types of vapor generating semi-solids include high viscous slurries. The solid or semi-solid vaporizable consumable may comprise a plant derived material, in particular tobacco.

Preferably, the solid or semi-solid vaporizable consumable comprises an aerosol-former. Examples of aerosol-formers include polyhydric alcohols and mixtures thereof such as glycerin or propylene glycol. Typically, the solid or semi-solid vaporizable consumable comprises an aerosol-former content of between approximately 5% and approximately 50% on a dry weight basis. Further preferably, the solid or semi-solid vaporizable consumable comprises an aerosol-former content of approximately 15% on a dry weight basis.

Upon heating, the solid or semi-solid vaporizable consumable may release volatile compounds. The volatile compounds may include nicotine or flavor compounds such as tobacco flavoring.

### BRIEF DESCRIPTION OF THE DRAWINGS

FIG. 1 shows a schematic demonstrating the loading of a vaporizer using a dispenser in accordance with an example of the first aspect of the invention.

FIG. 2 shows a illustrative 3D rendering of the vaporizer and dispenser used in FIG. 1.

FIG. 3 shows a schematic demonstrating the unloading and re-loading of a vaporizer using a dispenser in accordance with an example of a preferred assembly of the first embodiment of the first aspect of the invention.

FIG. 4 shows a schematic demonstrating the unloading of a vaporizer using a dispenser in accordance with an example of an alternate preferred assembly of the first embodiment of the first aspect of the invention.

FIG. 5 shows a schematic demonstrating the unloading of a vaporizer using a dispenser in accordance with an example of a preferred assembly of the second embodiment of the first aspect of the invention.

FIG. 6 shows a schematic demonstrating an additional embodiment of the loading of a vaporizer using a dispenser.

### DETAILED DESCRIPTION OF EMBODIMENTS OF THE INVENTION

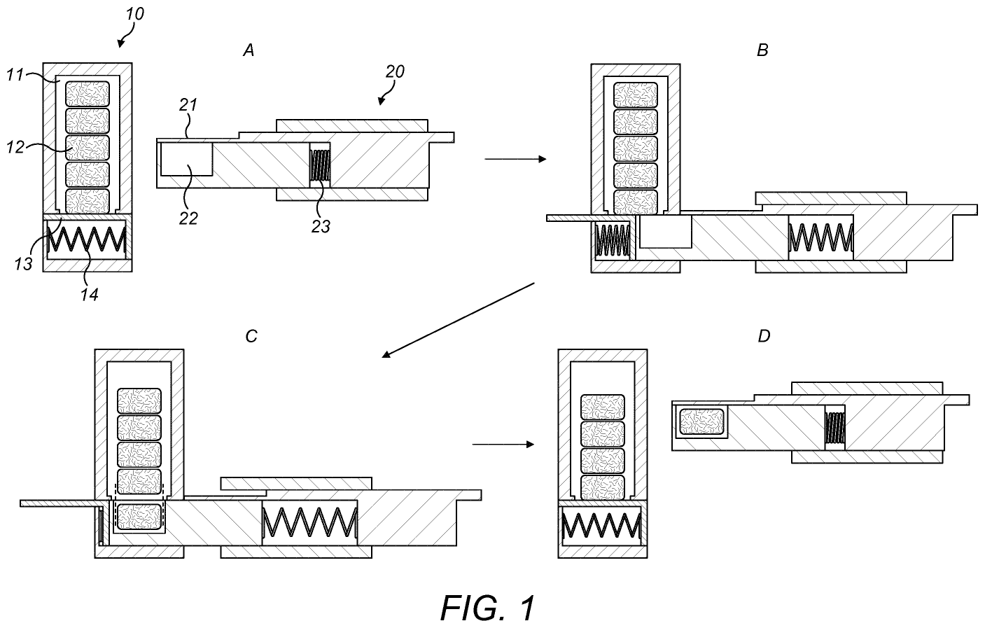

FIG. 1 schematically illustrates the loading of a vaporizer 20 using a dispenser 10 in accordance with an example of the first aspect of the invention. In this example, the vaporizer 20 is loaded with an unused consumable 12 from the dispenser 10.

The dispenser 10 includes a first store 11 for storing unused consumables 12, and a first lid 13 for closing the opening to the first store 11, whilst the vaporizer 20 includes a heating chamber 22, and a second lid 21 for closing the opening to the heating chamber 22.

The first lid 13 is arranged such that it is in mechanical communication with a first compression spring 14 such that when no external force is exerted upon the first lid 13 (e.g. in states A and D), the opening to the first store 11 is closed.

The second lid 21 is arranged such that it is in mechanical communication with a second compression spring 23 such that when no external force is exerted upon the second lid 21 (e.g. in states A and D), the opening to the heating chamber 22 is closed.

In state A, the dispenser 10 and the vaporizer 20 are in the first/fifth relative position.

Moving from state A to state B, it can be seen that engaging the vaporizer 20 with the dispenser 10 allows the first lid 13 and the second lid 21 to move from their respective closed positions.

Moving from state B to state C, it can be seen that the opening in the first store 11 is aligned with the opening in the heating chamber 22 such that an unused consumable 12 may be transferred from the first store 11 to the heating chamber 22 under gravity. In state C, the dispenser 10 and the vaporizer 20 are in the second/sixth relative position.

Moving from state C to state D, it can be seen that disengaging the vaporizer 20 from the dispenser 10 allows the first lid 13 and the second lid 21 to return to their respective closed positions. In state D, the dispenser 10 and the vaporizer 20 have returned to the first relative position, with an unused consumable 12 having been transferred from the first store 11 to the heating chamber 22.

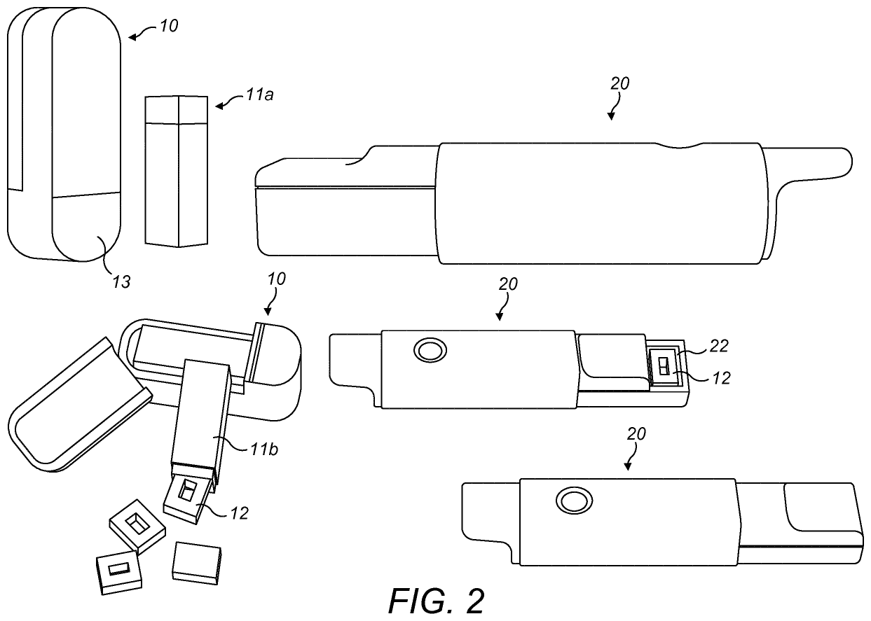

FIG. 2 shows a computer-generated rendering of the vaporizer and dispenser used in FIG. 1. In addition to the features mentioned for FIG. 1, a cartridge 11a for storing a detached replacement first store 11b is also shown. For example, a cartridge 11a containing a detached replacement first store 11b filled with unused consumables 12 may be used with the dispenser 10 of FIG. 1 such that the first store 11 is detached from the dispenser 10 and replaced by the detached replacement first store 11b as desired by the user.

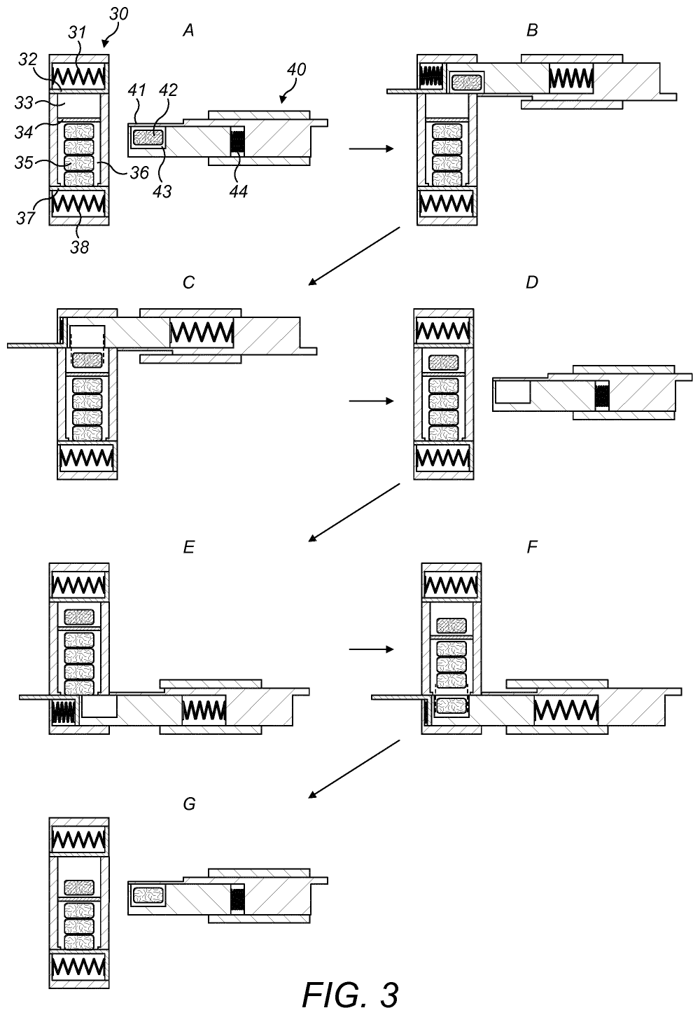

FIG. 3 schematically illustrates the unloading and re-loading of a vaporizer 40 using a dispenser 30 in accordance with an example of a preferred assembly of the first embodiment of the first aspect of the invention. In this example, the vaporizer 40 unloads a used consumable 42 into the dispenser 30, and is reloaded with an unused consumable 35 from the same dispenser 30.

The dispenser 30 includes a first store 36 for storing unused consumables 35, a first lid 37 for closing the opening to the first store 36, a second store 33 for storing used consumables 42, a second lid 32 for closing the opening to the second store 33, and a movable divider 34 separating the first store 36 from the second store 33, whilst the vaporizer 40 includes a heating chamber 43, and a third lid 41 for closing the opening to the heating chamber 43.

The first lid 37 is arranged such that it is in mechanical communication with a first compression spring 38 such that when no external force is exerted upon the first lid 37 (e.g. in states A-D and G), the opening to the first store 36 is closed.

The second lid 32 is arranged such that it is in mechanical communication with a second compression spring 31 such that when no external force is exerted upon the second lid 32 (e.g. in states A and D-G), the opening to the second store 33 is closed.

The third lid 41 is arranged such that it is in mechanical communication with a third compression spring 44 such that when no external force is exerted upon the third lid 41 (e.g. in states A, D, and G), the opening to the heating chamber 43 is closed.

In state A, the dispenser 30 and the vaporizer 40 are in the first/third/fifth relative position.

Moving from state A to state B, it can be seen that engaging the vaporizer 40 with the dispenser 30 allows the second lid 32 and the third lid 41 to move from their respective closed positions.

Moving from state B to state C, it can be seen that the opening in the heating chamber 43 is aligned with the opening in the second store 33 such that a used consumable 42 may be transferred from the heating chamber 43 to the second store 33 under gravity. In state C, the dispenser 30 and the vaporizer 40 are in the fourth/sixth relative position.

Moving from state C to state D, it can be seen that disengaging the vaporizer 40 from the dispenser 30 allows the second lid 32 and the third lid 41 to return to their respective closed positions. In state D, the dispenser 30 and the vaporizer 40 have returned to the first/third/fifth relative position, with a used consumable 42 having been transferred from the heating chamber 43 to the second store 33.

Moving from state D to state E, it can be seen that engaging the vaporizer 40 with the dispenser 30 allows the first lid 37 and the third lid 41 to move from their respective closed positions.

Moving from state E to state F, it can be seen that the opening in the first store 36 is aligned with the opening in the heating chamber 43 such that an unused consumable 35 may be transferred from the first store 36 to the heating chamber 43 under gravity. In state F, the dispenser 30 and the vaporizer 40 are in the second/sixth relative position.

Moving from state F to state G, it can be seen that disengaging the vaporizer 40 from the dispenser 30 allows the first lid 37 and the third lid 41 to return to their respective closed positions. In state D, the dispenser 30 and the vaporizer 40 have returned to the first/third/fifth relative position, with an unused consumable 35 having been transferred from the first store 35 to the heating chamber 43.

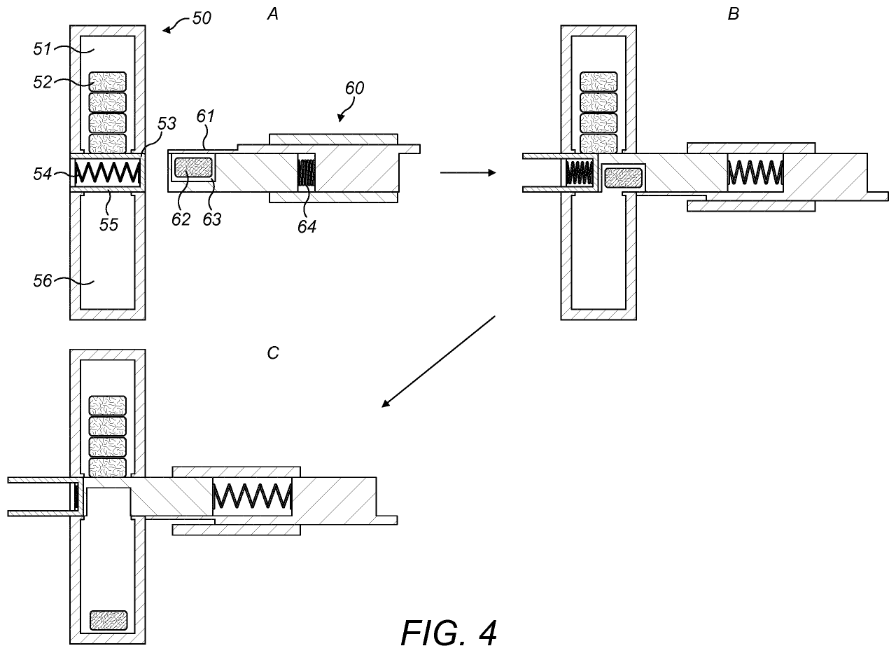

FIG. 4 schematically illustrates the unloading of a vaporizer 60 using a dispenser 50 in accordance with an example of an alternate preferred assembly of the first embodiment of the first aspect of the invention. In this example, the vaporizer 60 unloads a used consumable 62 into the dispenser 50. The mechanism for loading the vaporizer 60 with an unused consumable 52 works in the manner demonstrated in states A-D of FIG. 1.

The dispenser 50 includes a first store 51 for storing unused consumables 52, a first lid 53 for closing the opening to the first store 51, a second store 56 for storing used consumables 62, and a second lid 55 for closing the opening to the second store 56, whilst the vaporizer 60 includes a heating chamber 63, and a third lid 61 for closing the opening to the heating chamber 63.

The first lid 53 and second lid 55 are fixed to each other and arranged such that they are in mechanical communication with a first compression spring 54 such that when no external force is exerted upon first lid 53 and second lid 55 (e.g. in state A), the openings to the first store 51 and the second store 56 are closed.

The third lid 61 is arranged such that it is in mechanical communication with a second compression spring 64 such that when no external force is exerted upon the third lid 61 (e.g. in state A), the opening to the heating chamber 63 is closed.

In state A, the dispenser 50 and the vaporizer 60 are in the first/third/fifth relative position.

Moving from state A to state B, it can be seen that engaging the vaporizer 60 with the dispenser 50 allows the first lid 53, second lid 55, and the third lid 41 to move from their respective closed positions.

Moving from state B to state C, due to the orientation of the vaporizer 60, it can be seen that the opening in the heating chamber 63 is aligned with the opening in the second store 56 such that a used consumable 62 may be transferred from the heating chamber 63 to the second store 56 under gravity. In state C, the dispenser 50 and the vaporizer 60 are in the fourth/sixth relative position. Disengaging the vaporizer 60 from the dispenser 50 works in the manner demonstrated in states C-D of FIG. 2.

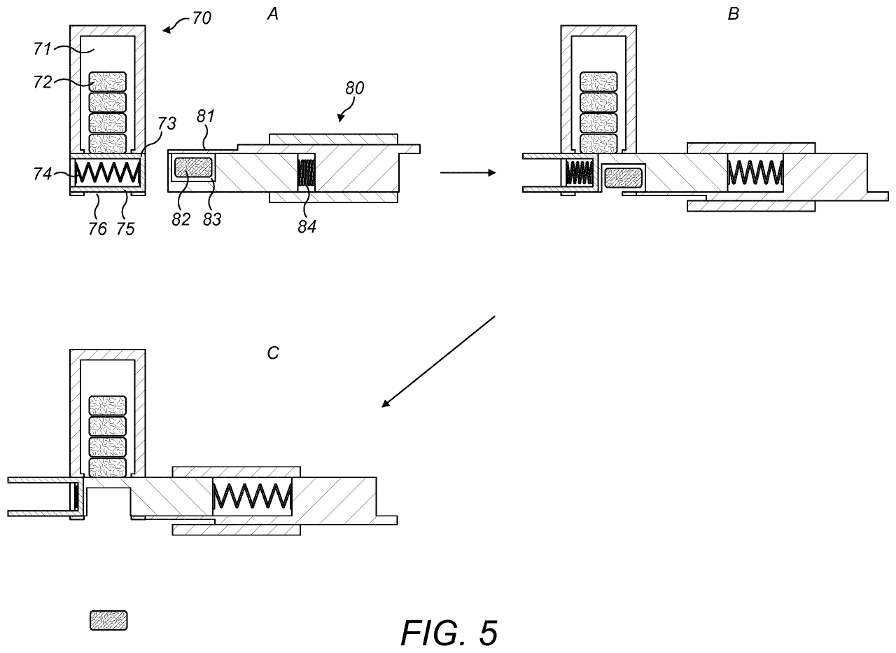

FIG. 5 schematically illustrates the unloading of a vaporizer 80 using a dispenser 70 in accordance with an example of a preferred assembly of the second embodiment of the first aspect of the invention. In this example, the vaporizer 80 unloads a used consumable 82 into the dispenser 70. The mechanism for loading the vaporizer 80 with an unused consumable 72 works in the manner demonstrated in states A-D of FIG. 1.

The dispenser 70 includes a first store 71 for storing unused consumables 72, a first lid 73 for closing the opening to the first store 71, a dispenser opening 76, and a second lid 75 for closing the dispenser opening 76, whilst the vaporizer 80 includes a heating chamber 83, and a third lid 81 for closing the opening to the heating chamber 83.

The first lid 73 and second lid 75 are fixed to each other and arranged such that they are in mechanical communication with a first compression spring 74 such that when no external force is exerted upon first lid 73 and second lid 75 (e.g. in state A), the openings to the first store 71 and the dispenser opening 76 are closed.

The third lid 81 is arranged such that it is in mechanical communication with a second compression spring 84 such that when no external force is exerted upon the third lid 81 (e.g. in state A), the opening to the heating chamber 83 is closed.

In state A, the dispenser 70 and the vaporizer 80 are in the first/third/fifth relative position.

Moving from state A to state B, it can be seen that engaging the vaporizer 80 with the dispenser 70 allows the first lid 73, second lid 75, and the third lid 81 to move from their respective closed positions.

Moving from state B to state C, due to the orientation of the vaporizer 80, it can be seen that the opening in the heating chamber 83 is aligned with the dispenser opening 76 such that a used consumable 82 may ejected from the heating chamber 83 (e.g. to a trash can) under gravity. In state C, the dispenser 70 and the vaporizer 80 are in the fourth/sixth relative position. Disengaging the vaporizer 80 from the dispenser 70 works in the manner demonstrated in states C-D of FIG. 2.

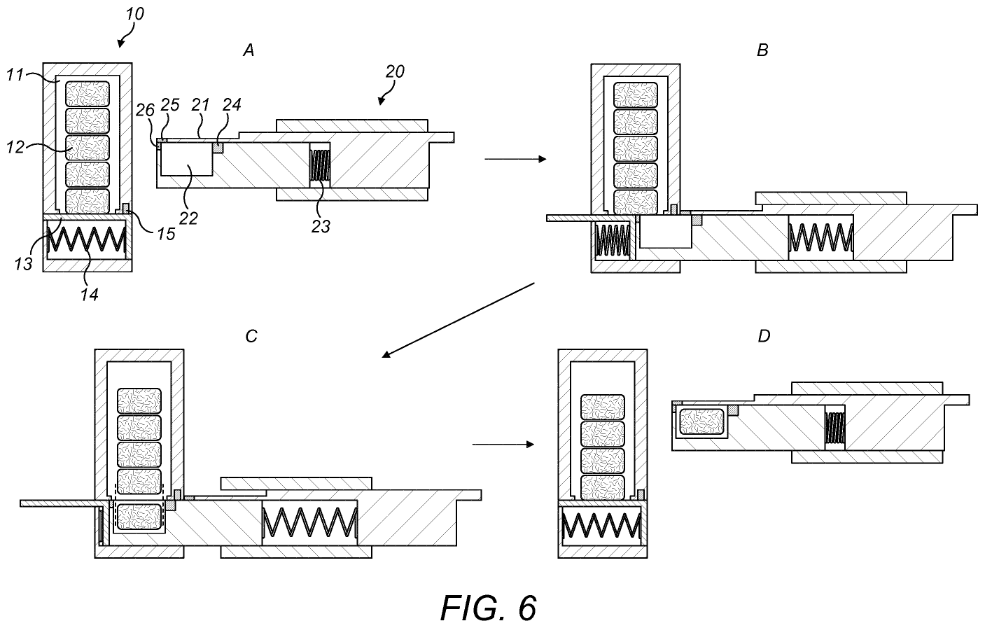

The dispensers and/or vaporizers described above may optionally also comprise one or more sensors for detecting a current state of the system and a control circuit for interpreting inputs from the one or more sensors. An example of such sensors is shown in FIG. 6 as a modification of FIG. 1.

For example, the system may comprise a first sensor 24 for detecting state C of FIG. 6 in which the opening in the first store 11 is aligned with the opening in the heating chamber 22. In other words, the first sensor 24 is configured to detect engagement of the vaporizer 20 with the dispenser 10.

Additionally or alternatively, the vaporizer 20 may comprise a second sensor 26 for detecting when the opening to the heating chamber 22 is closed as in state D. An edge event in a signal from the second sensor may be used by the control circuit to detect closure of the second lid 21.

If the vaporizer 20 is provided with sensors for both of states C and D, and a control circuit configured to detect a sequence of state C followed by state D, the vaporizer can detect when the heating chamber 22 has been loaded. This may, for example, be used to control activation of the vaporizer 20 to heat a consumable 12 in the heating chamber 22. A sensor and control circuit could equally be used in the dispenser, for example in order to count dispensing events and track how many unused consumables 12 remain in the dispenser.

Each of the sensor(s) is a proximity sensor for detecting when a particular relative positioning of components of the vaporizer 20 and/or dispenser 10 is achieved. For example, each of the sensors may be attached to a first component and configured to detect a detectable element attached to a second component. The sensor(s) may, for example, be conventional sensors or even switch mechanisms. However, the system preferably comprises one or more pairings of a magnet (detectable element) and a Hall effect sensor and/or pairings of a reflective surface (detectable element) and an optical sensor.

For example, a sensor for detecting state C may be implemented by providing a first magnet 15 in the dispenser 10 and providing a first Hall effect sensor 24 in the vaporizer 20. As shown in state C, when the dispenser and vaporizer are in state C, the first magnet 15 is near to the first Hall effect sensor 24, and can be detected by the first Hall effect sensor 24.

As another example, a sensor for detecting state D may be implemented by providing a second magnet 25 and a second Hall effect sensor 26 in the vaporizer 20, the second magnet 25 being attached to one of the second lid 21 and the heating chamber 22, and the second Hall effect sensor 26 being attached to the other of the second lid 21 and the heating chamber 22. As shown in FIG. 1, the magnet 25 and second Hall effect sensor 26 may be arranged such that they are close together when the opening to the heating chamber 22 is closed as in state D, and they are further apart when the opening to the heating chamber 22 is open as in state C. Therefore, by detecting when the magnetic field of second magnet 25 becomes stronger, the second Hall effect sensor 26 can detect that the opening to the heating chamber 22 is closed.

Advantageously, by arranging the first and second magnets 15, 25 such that they are close together in state C, Hall effect sensors can be used for detecting both of states C and D without requiring the first and second Hall effect sensors 24, 26 to distinguish between which of the first and second magnets 15, 25 is nearby. In one embodiment, the first magnet may be omitted and the Hall effect sensors 24, 26 may each detect the second magnet 25 at a different position of the second lid 21. In another embodiment, the first magnet 15 may be stronger than the second magnet 25, and the first Hall effect sensor 24 may be configured to be less sensitive than the second Hall effect sensor 26, such that the first Hall effect sensor 24 can distinguish the first magnet 15 (indicating that the vaporizer 20 is engaged with the dispenser 10) from the second magnet 25 (indicating only that the opening to the heating chamber 22 is open).

1. A dispenser for dispensing solid or semi-solid vaporizable consumables to a vaporizer, wherein
the dispenser comprises:
a first store for storing at least one solid or semi-solid vaporizable consumable therein, wherein the first store comprises an opening for dispensing a solid or semi-solid vaporizable consumable therethrough; and
a first lid, wherein the first lid is arranged such that it closes the opening in the first store when in a closed position;
wherein the dispenser is arranged such that, in use, engaging a vaporizer with the dispenser loads a heating chamber in the vaporizer with a solid or semi-solid vaporizable consumable stored within the first store by aligning the opening in the first store with an opening in the heating chamber.

2. The dispenser according to claim 1, wherein
the dispenser further comprises:
a second store having a capacity for storing at least one solid or semi-solid vaporizable consumable, wherein the second store comprises an opening for receiving a solid or semi-solid vaporizable consumable therethrough;
and, the dispenser is arranged such that, in use, engaging a vaporizer with the dispenser to unload a solid or semi-solid vaporizable consumable stored within a heating chamber in the vaporizer aligns the opening in the second store with an opening in the heating chamber.

3. The dispenser according to claim 2, wherein the first store and the second store are conjoined such that the opening of the second store is at an end of the body of the conjoined stores distal to the opening of the first store.

4. The dispenser according to claim 3, wherein the conjoined stores comprise a movable divider for defining the first store and the second store such that when the first store is full of unused solid or semi-solid vaporizable consumables the first store is larger than the second store, and when there are no unused solid or semi-solid vaporizable consumables within the first store the second store is larger than the first store.

5. The dispenser according to claim 1, wherein
the dispenser further comprises:
an opening for ejecting a solid or semi-solid vaporizable consumable therethrough;
wherein the dispenser is arranged such that, in use, engaging a vaporizer with the dispenser to unload a solid or semi-solid vaporizable consumable stored within a heating chamber in the vaporizer aligns the opening in the dispenser with an opening in the heating chamber.

6. The dispenser according to claim 2, wherein the dispenser further comprises a second lid, wherein the second lid is arranged such that it closes the opening in the second store or the opening in the dispenser when in a closed position.

7. The dispenser according to claim 1, wherein the first store and/or the second store are either independently or simultaneously detachable from the dispenser housing.

8. The dispenser according to claim 1, wherein the dispenser further comprises an indicator or a window for determining the number of solid or semi-solid vaporizable consumables stored in the first store.

9. The dispenser according to claim 1, comprising one or more sensors configured to detect when the dispenser loads the heating chamber in the vaporizer with the solid or semi-solid vaporizable consumable.

10. The dispenser according to claim 1, comprising a detectable element configured to be detected by a sensor of the vaporizer when the vaporizer is engaged with the dispenser.

11. A vaporizer for use in combination with the dispenser according to claim 1, wherein
the vaporizer comprises:
a heating chamber for receiving and heating a solid or semi-solid vaporizable consumable to generate vapor therefrom, the heating chamber comprising an opening for receiving and ejecting a solid or semi-solid vaporizable consumable therethrough; and
a third lid, wherein the third lid is arranged such that it closes the opening in the heating chamber when in a closed position;
wherein the vaporizer is arranged such that, in use, engaging the vaporizer with the dispenser loads the heating chamber with a solid or semi-solid vaporizable consumable stored within the first store by aligning the opening in the first store with the opening in the heating chamber to receive the consumable.

12. The vaporizer according to claim 11, wherein the third lid is locked in its closed position until a corresponding switch on the vaporizer is activated thereby allowing the third lid to open.

13. The vaporizer according to claim 11, comprising one or more sensors configured to detect when the dispenser loads the heating chamber in the vaporizer with the solid or semi-solid vaporizable consumable.

14. The vaporizer according to claim 13, wherein the one or more sensors comprises a first sensor configured to detect a detectable element of the dispenser when the vaporizer is engaged with the dispenser.

15. The vaporizer according to claim 13, wherein the one or more sensors comprises a second sensor configured to detect that the third lid is in the closed position.

16. The vaporizer according to claim 14, comprising a control circuit configured to detect that the dispenser has loaded the heating chamber in response to the second sensor detecting that the third lid is in the closed position after the first sensor detects that the vaporizer is engaged with the dispenser.

17. The vaporizer according to claim 11, wherein each lid is biased in a closed position when no external force is being exerted upon said lids.

18. A solid or semi-solid vaporizable consumable supply system comprising a vaporizer and the dispenser according to claim 1, wherein the vaporizer and the dispenser are arranged such that, in use, engaging the vaporizer with the dispenser biases the first lid and the third lid into their respective open positions and aligns the opening in the heating chamber with the opening in the first store thereby allowing a solid or semi-solid vaporizable consumable stored within the first store to be received from the first store into the heating chamber.

19. The supply system according to claim 18, wherein the vaporizer and the dispenser are arranged such that, in use, engaging the vaporizer with the dispenser to unload the heating chamber either:
biases the second lid and the third lid into their respective open positions and aligns the opening in the heating chamber with the opening in the second store thereby allowing a solid or semi-solid vaporizable consumable stored within the heating chamber to be received from the heating chamber into the second store; or
biases the first and/or second lid(s) and the third lid into their respective open positions and aligns the opening in the heating chamber with the opening in the dispenser thereby allowing a solid or semi-solid vaporizable consumable stored within the heating chamber to be ejected from the system.

20. The supply system according to claim 18, wherein the vaporizer and the dispenser are arranged such that a solid or semi-solid vaporizable consumable may be received from the first store into the heating chamber by gravity.

21. The supply system according to claim 18, wherein the vaporizer and the dispenser are arranged such that a solid or semi-solid vaporizable consumable may be received from the heating chamber into the second store or ejected from the system by gravity.
